(19) United States
(12) Patent Application Publication
(10) Pub. No.: US 2025/0295895 A1
(43) Pub. Date: Sep. 25, 2025

(54) TELESCOPING VASCULAR SHEATH SYSTEMS AND METHODS

(71) Applicant: Silk Road Medical, Inc., Sunnyvale, CA (US)

(72) Inventors: Sumaira Macdonald, Sunnyvale, CA (US); Blaine Schneider, Sunnyvale, CA (US); Brad Steele, Sunnyvale, CA (US); William Whealon, Sunnyvale, CA (US); Mark C. Page, Sunnyvale, CA (US)

(73) Assignee: Silk Road Medical, Inc., Sunnyvale, CA (US)

(21) Appl. No.: 18/868,987

(22) PCT Filed: May 25, 2023
(86) PCT No.: PCT/US2023/023603

§ 371 Date: Nov. 25, 2024

Related U.S. Application Data
(60) Provisional application No. 63/346,265, filed on May 26, 2022.
(60) Provisional application No. 63/482,881, filed on Feb. 2, 2023.

(51) Int. Cl. A61M 25/10; A61M 25/00; A61M 29/00

(52) U.S. Cl. CPC A61M 25/10; A61M 25/0023; A61M 29/00; A61M 2025/1093; A61M 2205/3334; A61M 2210/12

(57) ABSTRACT

Various embodiments and related methods of a telescoping vascular sheath are disclosed. The telescoping vascular sheath can include a plurality of sheath segments each having a tapered distal section and a proximal section. Each tapered distal section can include a distal opening surface and each proximal section can include an outer proximal surface. Each sheath segment of the plurality of sheath segments can be slidably engaged with at least one adjacent sheath segment along one or more of the distal opening surfaces and the outer proximal surface. The telescoping vascular sheath can also include a working passageway extending through the plurality of sheath segments and along a longitudinal axis of the telescoping vascular sheath. The working passageway can provide access to an inner lumen of the vessel. In some embodiments, a blood flow system is disclosed.

### CROSS-REFERENCE TO RELATED APPLICATION

The current application claims priority under 35 U.S.C. § 119(e) to U.S. Provisional patent application Ser. Nos. 63/346,265, filed on May 26, 2022, and entitled “TELESCOPING VASCULAR SHEATH”, and 63/482,881, filed on Feb. 2, 2023, and entitled “TELESCOPING VASCULAR SHEATH”, which are incorporated by reference herein in their entirety.

### BACKGROUND

Various medical procedures involve accessing inner lumens of vessels. Sheaths or catheters can provide a conduit from an extracorporeal space to the inside of the body. These devices may be used to insert tools into the body or provide therapy during various procedures and through a variety of access sites (e.g. surgical, percutaneous, or existing body orifices). In general, it is desired to create the smallest entryway possible that accommodates a given treatment to reduce wound healing time and infection risk, speed recovery, and improve patient outcomes and comfort, when compared to larger entry sites.

For example, mechanical thrombectomy can be an important tool when treating stroke. Mechanical thrombectomy may include the use of aspiration catheters, stent retrievers, or the combination of the two. To perform these treatments, physicians can access the vasculature using successively larger sheaths until the desired size has been reached to accommodate and support the chosen mechanical thrombectomy treatment. Exchanging sheaths for an appropriate sized sheath can risk loss of access at the intended entry site, can be time consuming, and increase treatment costs. As such, methods and devices for reducing or preventing such consequences due to exchanging sheaths is desired.

### SUMMARY

Aspects of the current subject matter include embodiments of a telescoping vascular sheath for insertion into a vessel. In one aspect, the telescoping vascular sheath can include a plurality of sheath segments each having a tapered distal section and a proximal section. Each tapered distal section can include a distal opening surface and each proximal section can include an outer proximal surface. Additionally, each sheath segment of the plurality of sheath segments can be slidably engaged with at least one adjacent sheath segment along one or more of the distal opening surfaces and the outer proximal surface. The telescoping vascular sheath can further include a working passageway extending through the plurality of sheath segments and along a longitudinal axis of the telescoping vascular sheath. The working passageway can provide access to an inner lumen of the vessel.

In some variations one or more of the following features can optionally be included in any feasible combination. For example, each sheath segment of the plurality of sheath segments can be independently moveable along the longitudinal axis. The proximal section can include a tubular shape that extends a distance along the longitudinal axis. The tapered distal section can include a distal opening defined by the distal opening surface. The distal opening surface can be approximately linear and parallel to the longitudinal axis. The distal opening surface can be approximately linear and parallel to the longitudinal axis. The working passageway can be defined by a first sheath segment of the plurality of sheath segments, and the distal opening surface of the first sheath segment can be smaller in diameter compared to distal opening surfaces associated with each of the other sheath segments of the plurality of sheath segments.

In some embodiments, the distal opening surface of a second sheath segment of the plurality of sheath segments can form a sliding fit along the proximal section of the first sheath segment. The tapered distal section can provide atraumatic insertion of the telescoping vascular sheath into tissue and inner lumen of a vessel. The overall length of each sheath segment of the plurality of sheath segments can be approximately 5 centimeters (cm) to approximately 200 cm. The diameter of the distal opening can be approximately 0.01 in to approximately 20 F. The telescoping vascular can include a first sheath positioned closest to a longitudinal axis of the telescoping vascular sheath. The first sheath can include the proximal section having a smaller diameter and an overall length that is longer compared to other sheath segments of the telescoping vascular sheath.

In some embodiments, the telescoping vascular sheath can include an outer sheath segment of the plurality of sheath segments positioned furthest away from the longitudinal axis of the telescoping vascular sheath. The outer sheath segment can include a first proximal section that is greater in diameter and shorter in length compared to proximal sections associated with each of the other sheath segments of the plurality of sheath segments. The telescoping vascular sheath can further include a valve positioned at a proximal end of at least one sheath segment of the plurality of sheath segments. The telescoping vascular sheath can further include a port positioned at a proximal end of at least one sheath segment of the plurality of sheath segments. The port can include a fluid pathway that is in fluid communication with the working passageway. The telescoping vascular sheath can include at least one sheath segment of the plurality of sheath segments having an inflatable balloon. The inflatable balloon can expand radially out from the proximal section when in an inflated state. At least one sheath segment of the plurality of sheath segments can provide atraumatic dilation of tissue and atraumatic dilation into the vessel.

In another interrelated aspect of the current subject matter, a method of a telescoping vascular sheath includes advancing a first sheath segment of a plurality of sheath segments of the telescoping vascular sheath into an inner lumen of a vessel. The method can further include advancing a second sheath segment of a plurality of sheath segments of the telescoping vascular sheath into the inner lumen of the vessel. The telescoping vascular sheath can include the plurality of sheath segments each having a tapered distal section and a proximal section. Each tapered distal section can include a distal opening surface and each proximal section can include an outer proximal surface. Additionally, each sheath segment of the plurality of sheath segments can be slidably engaged with at least one adjacent sheath segment along one or more of the distal opening surfaces and the outer proximal surface. The telescoping vascular sheath can further include a working passageway extending through the plurality of sheath segments and along a longitudinal axis of the telescoping vascular sheath. The working passageway can provide access to an inner lumen of the vessel. The method can further include slidably translating the first sheath segment relative to the second sheath segment.

In some variations of the method one or more of the following features can optionally be included in any feasible combination. For example, each sheath segment of the plurality of sheath segments can be independently moveable along the longitudinal axis. The proximal section can include a tubular shape that extends a distance along the longitudinal axis. The tapered distal section can include a distal opening defined by the distal opening surface. The distal opening surface can be approximately linear and parallel to the longitudinal axis. The distal opening surface can be approximately linear and parallel to the longitudinal axis. The working passageway can be defined by a first sheath segment of the plurality of sheath segments, and the distal opening surface of the first sheath segment can be smaller in diameter compared to distal opening surfaces associated with each of the other sheath segments of the plurality of sheath segments.

In some embodiments, the distal opening surface of a second sheath segment of the plurality of sheath segments can form a sliding fit along the proximal section of the first sheath segment. The tapered distal section can provide atraumatic insertion of the telescoping vascular sheath into tissue and inner lumen of a vessel. The overall length of each sheath segment of the plurality of sheath segments can be approximately 5 centimeters (cm) to approximately 200 cm. The diameter of the distal opening can be approximately 0.01 in to approximately 20 F. The telescoping vascular can include a first sheath positioned closest to a longitudinal axis of the telescoping vascular sheath. The first sheath can include the proximal section having a smaller diameter and an overall length that is longer compared to other sheath segments of the telescoping vascular sheath.

In some embodiments, the telescoping vascular sheath can include an outer sheath segment of the plurality of sheath segments positioned furthest away from the longitudinal axis of the telescoping vascular sheath. The outer sheath segment can include a first proximal section that is greater in diameter and shorter in length compared to proximal sections associated with each of the other sheath segments of the plurality of sheath segments. The telescoping vascular sheath can further include a valve positioned at a proximal end of at least one sheath segment of the plurality of sheath segments. The telescoping vascular sheath can further include a port positioned at a proximal end of at least one sheath segment of the plurality of sheath segments. The port can include a fluid pathway that is in fluid communication with the working passageway. The telescoping vascular sheath can include at least one sheath segment of the plurality of sheath segments having an inflatable balloon. The inflatable balloon can expand radially out from the proximal section when in an inflated state. At least one sheath segment of the plurality of sheath segments can provide atraumatic dilation of tissue and atraumatic dilation into the vessel.

In another aspect, a blood flow system is disclosed that includes a first arterial access device for accessing a first artery, a second arterial access device for accessing a second artery, and an arterial return device for communicating with a third artery. The arterial return device can be in fluid communication with the first arterial access device and the second arterial access device.

In some variations one or more of the following features can optionally be included in any feasible combination. For example, one or more of the first arterial access device, the second arterial access device, and the arterial return device can include a telescoping vascular sheath. In some embodiments, the blood flow system can further include a splitter that directs fluid flow from the arterial return device to both the first arterial access device and the second arterial access device. In some embodiments, the blood flow system can further include a flow control element that controls rate of flow between the arterial return device and the splitter.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 illustrates a partially exploded side view of an embodiment of an access device including a sheath for providing access to an inner lumen of a vessel.

FIG. 2 illustrates a side partial view of the access device of FIG. 1 including a sheath stopper.

FIG. 3 illustrates an example of the access system of FIG. 1 accessing an inner lumen of a vessel.

FIG. 4A illustrates a side view of an embodiment of a telescoping vascular sheath including a plurality of sheath segments.

FIG. 4B illustrates a side cross-section view of the telescoping vascular sheath of FIG. 4A.

FIG. 4C illustrates a side cross-section view of an embodiment of one of the plurality of sheath segments of the telescoping vascular sheath of FIG. 4A.

FIG. 4D illustrates an example of the telescoping vascular sheath of FIG. 4A accessing an inner lumen of a vessel.

FIG. 4E illustrates a side cross-section view of another embodiment of one of the plurality of sheath segments of the telescoping vascular sheath of FIG. 4A including an inflatable balloon.

FIG. 5 is a schematic illustration of a blood flow system including at least one arterial access device that accesses the left common carotid artery and at least one arterial access device that accesses the right common carotid artery via a transcervical approach and an arterial return device that communicates with an artery, such as a femoral artery.

When practical, similar reference numbers denote similar structures, features, or elements.

### DETAILED DESCRIPTION

The disclosed methods, apparatus, and systems are directed to various embodiments of a telescoping vascular sheath that can efficiently and effectively create access into an inner lumen of a vessel, as well as form a working passageway through tissue and into the inner lumen, such as for performing a variety of procedures. The telescoping vascular sheaths described herein can include a plurality of sheath segments that are independently moveable relative to each other to allow the telescoping vascular sheath to form a variety of configurations. For example, each sheath segment can have different dimensions (e.g., outer and inner diameters, lengths, etc.) thereby allowing various configurations of the telescoping vascular sheath by positioning each sheath segment at different positions relative to each other, as well as including and removing one or more sheath segments. As such, the telescoping vascular sheath embodiments described herein can be configured to create access through tissue and into the lumen of a vessel, as well as form a working passageway through tissue and/or into the vessel that is appropriately sized for performing desired procedures. For example, in some embodiments at least one sheath segment (e.g., the largest diameter sheath segment) can be sized for providing dilation through the tissue, and not for insertion into the vessel. Such customized sizing of the telescoping vascular sheath can reduce wound healing time and infection risk, speed recovery, and improve patient outcomes and comfort.

The telescoping vascular sheath can also allow for efficient changing of configurations during a procedure, such as transitioning from a smaller diameter sheath segment positioned at a distal end of the telescoping vascular sheath to a larger diameter sheath segment. For example, a user may want the smaller diameter sheath segment positioned at the distal end during insertion of the telescoping vascular sheath into a vessel. Additionally, once entry into the vessel has been made by at least the smaller diameter sheath segment, the user may want to increase the working passageway through the telescoping vascular sheath in order to insert one or more devices through the working passageway and into the inner lumen of the vessel. Such transitioning to a larger working passageway can be efficiently achieved with the telescoping vascular sheath without having to retract the telescoping vascular sheath out of the inner lumen of the vessel.

For example, the smaller diameter sheath segment can be slid in a proximal direction to allow a sheath segment having a larger diameter to become positioned at the distal end. Additionally, the smaller diameter sheath segment can be removed and uncoupled from the remaining sheath segments that are slidably coupled together to form the working passageway. With the smaller diameter sheath removed and uncoupled, the working passageway can have a larger diameter (e.g., the inner diameter of the smallest remaining sheath segment). As such, the telescoping vascular sheath can prevent the need for multiple sheath exchanges thereby reducing the risks of lost access to the inner lumen of the vessel (which can require re-puncture or re-access to the inner lumen), as well as expedite treatment time, and reduce procedural costs.

In some embodiments, the telescoping vascular sheath can include a plurality of sheath segments that can be sized to assist with performing a variety of procedures, including cardiovascular, neurovascular, ophthalmic, gastrointestinal, and urological procedures. As such, the telescoping vascular sheath can be used with a variety of vasculature, including the common femoral, subclavian, carotid, jugular, inferior vena cava, etc., without departing from the scope of this disclosure. Furthermore, the telescoping vascular sheath can be used with one or more of a variety of access systems, including the exemplary access system described herein.

In some embodiments, a blood flow system is described. The blood flow system can include a first arterial access device that accesses a first vessel (e.g., the left common carotid artery) and a second arterial access device that accesses a second vessel (e.g., the right common carotid artery), such as via a transcervical approach. Additionally, the blood flow system can include an arterial return device that communicates with an artery, such as a femoral artery. The arterial return device can be in fluid communication with the first arterial access device and the second arterial access device. In some embodiments, any one or more of the first arterial access device, the second arterial access device, and the arterial return device can include embodiments of the telescoping vascular sheath described herein.

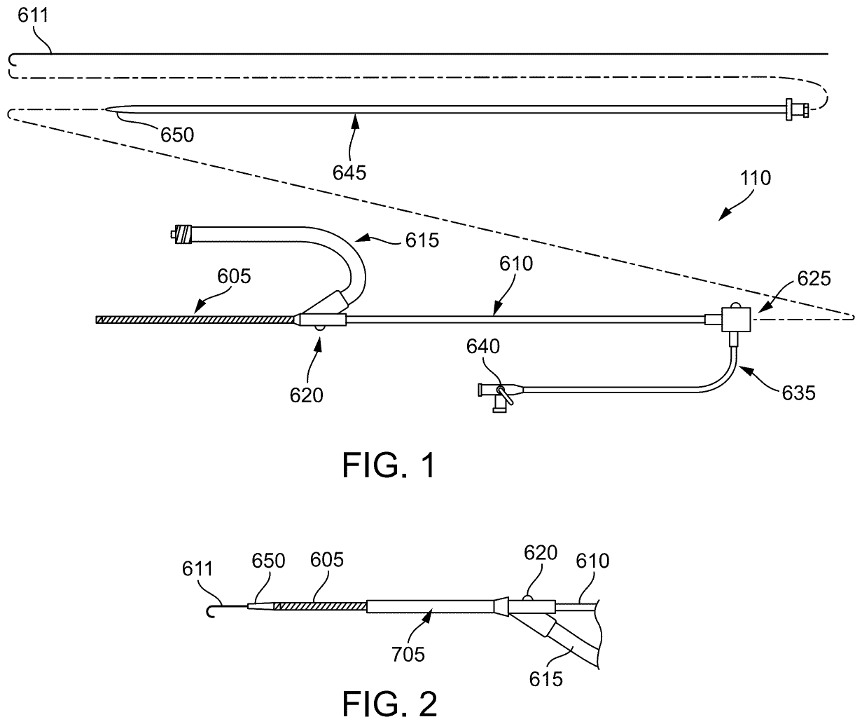

FIG. 1 shows an exemplary embodiment of an access system 110 including a distal sheath 605, a proximal extension 610, a flow line 615, an adaptor or Y-connector 620, and a hemostasis valve 625. The access system 110 may also comprise a dilator 645 with a tapered tip 650 and an introducer guide wire 611. The access system 110 together with the dilator 645 and introducer guidewire 611 can be used together to gain access to a vessel. Features of the access system may be optimized for access into a variety of vessels, such as via transfemoral and transcarotid access.

The distal sheath 605 can be adapted to be introduced through an incision or puncture in a wall of a vessel (e.g., femoral artery, common carotid artery, etc.), either an open surgical incision or a percutaneous puncture established, for example, using the Seldinger technique. As shown in FIG. 1, the proximal extension 610, which includes an elongated body, can have an inner lumen that is contiguous with an inner lumen of the sheath 605. The lumens can be joined by the Y-connector 620 that also connects a lumen of the flow line 615 to the sheath. For example, the flow line 615 can connect to and form a first leg of a retrograde shunt.

A flush line 635 can be connected to the side of the hemostasis valve 625 and can have a stopcock 640 at its proximal or remote end. The flush-line 635 can allow for the introduction of saline, contrast fluid, or the like, during the procedures. The flush line 635 can also allow pressure monitoring during the procedure. A dilator 645 having a tapered distal end 650 can be provided, for example, to facilitate introduction of the distal sheath 605 into the vessel. The dilator 645 can be introduced through the hemostasis valve 625 so that the tapered distal end 650 extends through the distal end of the sheath 605, as best seen in FIG. 2. The dilator 645 can have a central lumen to accommodate a guide wire. Typically, the guide wire is placed first into the vessel, and the dilator/sheath combination travels over the guide wire as it is being introduced into the vessel. Optionally, a sheath stopper 705 such as in the form of a tube may be provided that is coaxially received over the exterior of the distal sheath 605, as also seen in FIG. 2.

The access system 110 can provide access to an inner lumen of a vessel, such as for performing at least part of any number of a variety of procedures. For example, during a transcarotid artery revascularization (TCAR) procedure, the arterial sheath 605 can be inserted into the common carotid artery (CCA) of the patient. To achieve reverse flow of blood, the CCA may be occluded to stop antegrade blood flow from the aorta through the CCA. Flow through the CCA can be occluded with an external vessel loop or tape, a vascular clamp, an internal occlusion member such as a balloon, or other type of occlusion means. One or more of a variety of blood flow control can be used during a procedure using the access system 110.

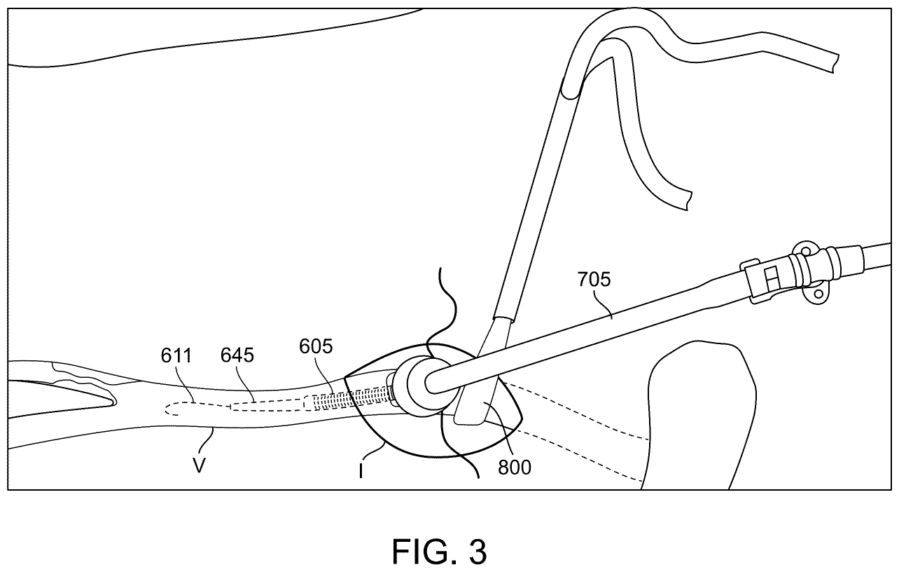

FIG. 3 illustrates an embodiment of an access system 110 including an embodiment of the sheath 605 inserted within a vessel V, such as the carotid artery, exposed through an incision I. For example, the sheath stopper 705 can be used with the sheath 605 to assist with guiding and positioning the sheath 605 within the vessel V, as shown in FIG. 3. The sheath guidewire 611 and dilator 645 can protrude out from a distal opening of the sheath 605. For example, manual occlusion of the vessel V by a clinician at an occlusion location proximal to the distal tip of the sheath 605 may be provided from the outside of the vessel V using a vascular clamp 800, such as a Rummel tourniquet or vessel loop positioned proximal to the sheath insertion site. Occlusion of the vessel can form a closed section of the vessel along which blood is prevented from flowing. Once the target vessel V has been treated by the operator, the vascular clamp 800 can be released permitting resumption of antegrade blood flow. One or more of a variety of ways can be used to occlude the vessel during a procedure without departing from the scope of this disclosure.

Various embodiments of a telescoping vascular sheath that can be used with a variety of access systems, including the access system 110 described herein, are described in detail below. As will be described in greater detail below, the telescoping vascular sheaths described herein can be configured to form a variety of different sizes, such as to form an appropriate working passageway for performing a desired procedure, thereby reducing wound healing time and infection risk, speed recovery, and improve patient outcomes and comfort. Additionally, the telescoping vascular sheaths described herein can prevent the need for multiple sheath exchanges thereby reducing the risks of lost access to the inner lumen of the vessel (which can require re-puncture or re-access to the inner lumen), as well as expedite treatment time, and reduce procedural costs.

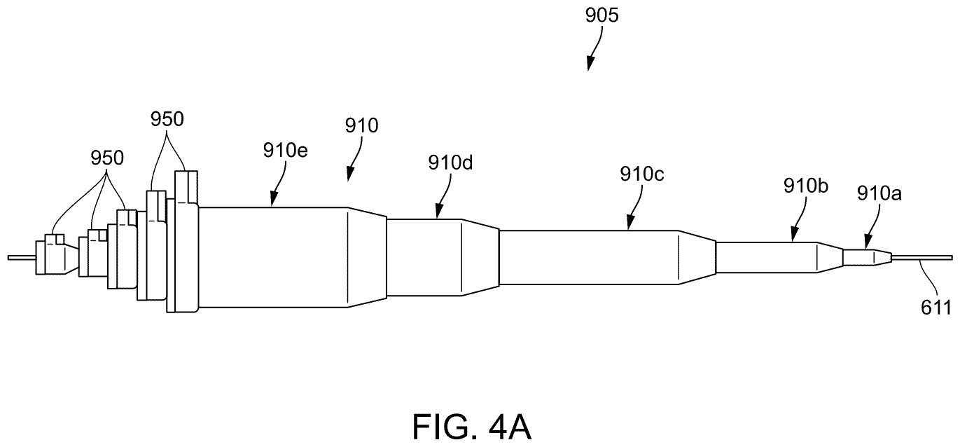

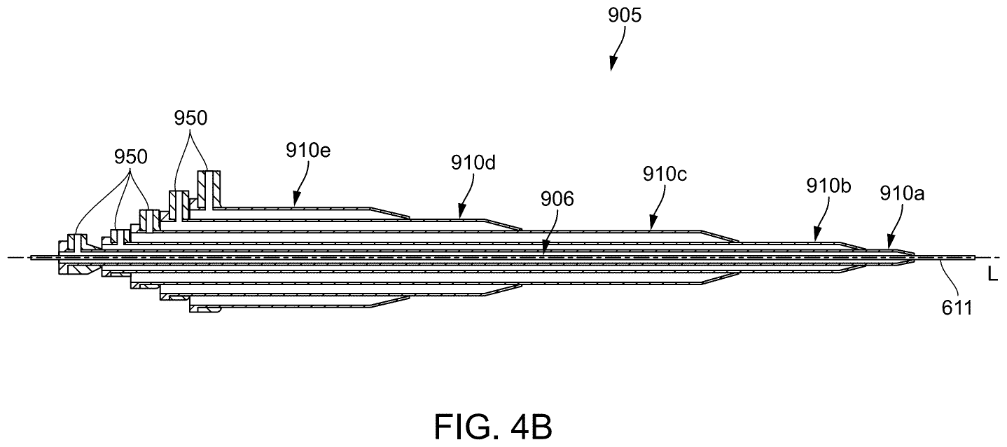

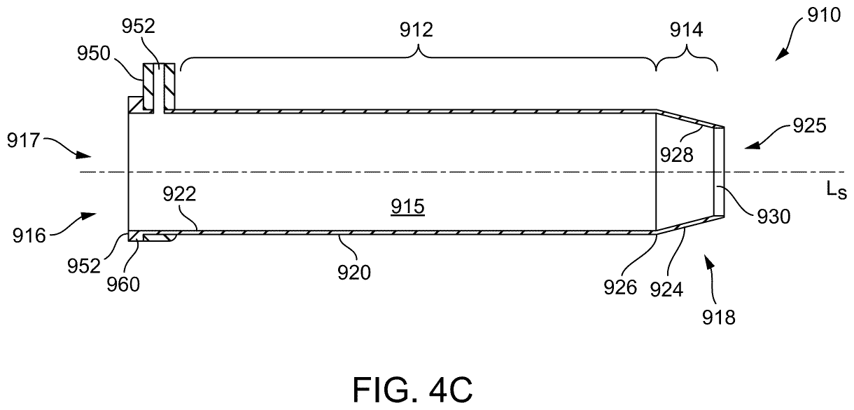

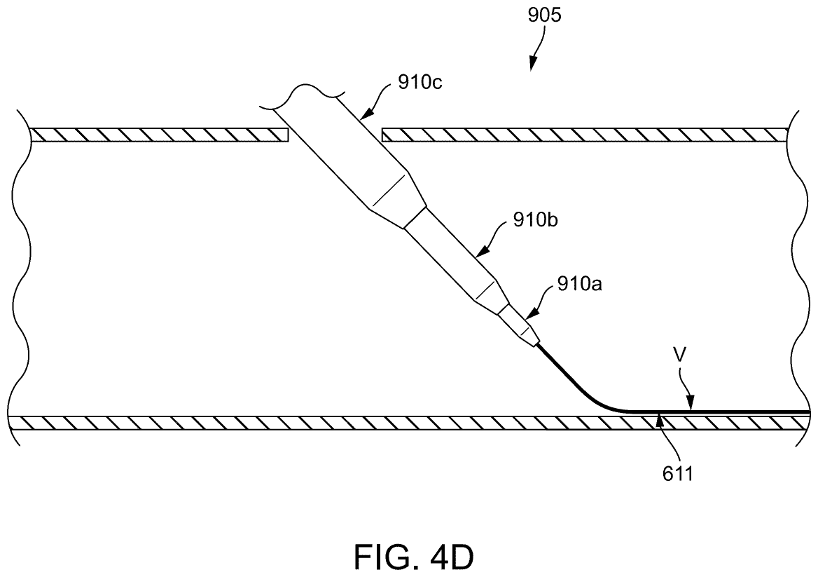

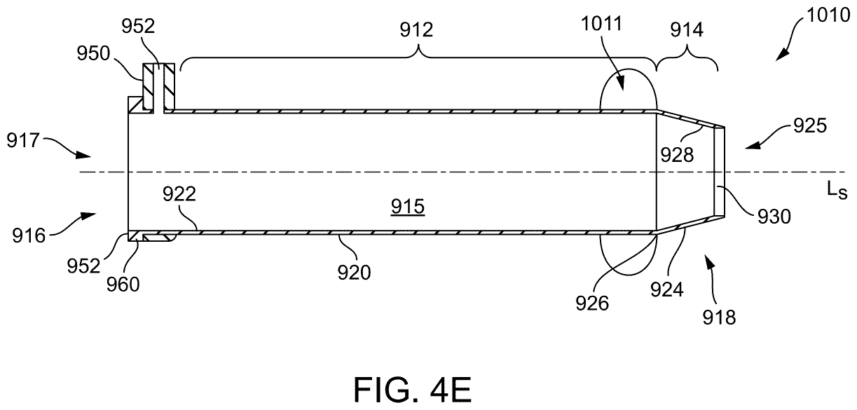

FIGS. 4A-4D illustrate an embodiment of a telescoping vascular sheath 905 that is configured to be partly inserted into tissue and/or vasculature for assisting with forming a working passageway 906, such as into the inner lumen of a vessel. As shown in FIG. 4A, the telescoping vascular sheath 905 can include a plurality of concentric sheath segments 910 that are slidably coupled together such that they can independently move relative to each other. The telescoping vascular sheath 905 can include two or more sheath segments 910, such as a first sheath segment, 910a, a second sheath segment 910b, a third sheath segment 910c, a fourth sheath segment 901d, and a fifth sheath segment 901e, as shown in FIGS. 4A and 4B. During use, any one or more of the sheath segments 910 can be used for assisting with performing a procedure, such as two or more adjacent sheath segments 910. Each sheath segment 910 can slidably engage at least one adjacent sheath segment 910 of the telescoping vascular sheath 905 and translate along a longitudinal axis L of the telescoping vascular sheath 905, as shown in FIG. 4B. Additionally, each sheath segment 910 can independently translate along the longitudinal axis L relative to one or more other sheath segments 910 of the telescoping vascular sheath 905.

As shown in FIG. 4C, each sheath segment 910 (e.g., sheath segments 910a, 901b, 910c, 910d, and 910e) can include a proximal section 912 and a tapered distal section 914 that extends from a distal end of the proximal section 912. Additionally, each sheath segment 910 can include a sheath segment passageway 915 or through hole that extends between a proximal opening 917 at a proximal end 916 and a distal opening 925 at a distal end 918 of each sheath segment 910. As shown in FIG. 4C, the proximal section 912 can include a tubular shape that extends approximately linearly along a longitudinal axis Ls of the sheath segment 910. The proximal section 912 can include an outer proximal surface 920 and an inner proximal surface 922. The inner proximal surface 922 can define a proximal portion of the sheath segment passageway 915, which can be tubular in shape. The tapered distal section 914 can include a tapered wall 924 that extends at an angle between a distal end of the proximal section 912 and the distal opening 925. The tapered distal section 914 can include an outer distal surface 926 and an inner distal surface 928 that extend along opposing sides of the tapered wall 924. The inner distal surface 928 can define at least a part of a distal portion of the sheath segment passageway 915. A distal end of the tapered wall 924 can include a distal opening surface 930 that defines the distal opening 925. The distal opening 925 can also define a part of the sheath segment passageway 915.

As shown in FIG. 4C, the distal opening surface 930 can be approximately linear and parallel to the longitudinal axis of the sheath 910. The distal opening surface 930 can be sized to allow the distal opening surface 930 to have a sliding fit along the outer proximal surface 920 of a smaller sheath segment 910. For example, the first sheath segment 910a can be smaller and extend along the sheath segment passageway 915 of the second sheath segment 910b, as shown in FIGS. 4A and 4B. The distal opening surface 930 of the second sheath segment 910b can have a sliding fit with the proximal section 912 of the first sheath segment 910a. This can allow two adjacent sheath segments 910 (e.g., the first sheath segment 910a and second sheath segment 910b) to independently and slidably translate relative to each other. For example, the first sheath segment 910a can be slidably translated along the longitudinal axis L to form a desired length between distal ends 918 or distal openings 925 of the first sheath segment 910a and the second sheath segment 910b.

The telescoping vascular sheath 905 can include any number of sheath segments 910 that each have a variety of shapes and sizes. For example, each sheath segment 910 can have different dimensions, such as to allow slidable coupling between the sheath segments 910. In some embodiments, at least some of the sheath segments 910 can provide specific functions, such as perform dilation, perform aspiration, perform as an introducer, etc. Additionally, one or more sheath segments 910 can provide more than one function, such as change functions based on a procedural step. For example, a sheath segment 910 can provide support for a microcatheter during tracking and also serve as an aspiration catheter, such as once a target anatomy is reached and smaller sheath segments 910 have been removed/uncoupled from the telescoping vascular sheath 905.

In some embodiments, the smallest sheath segment (e.g., the first sheath segment 901a) can have the smallest outer proximal surface diameter, which can have a sliding fit with the distal opening surface 930 of the adjacent sheath segment (e.g., the second sheath segment 910b). As such, the second sheath segment 910b can slidably translate along the outer proximal surface 920 of the first sheath segment 910a such as to allow the first sheath segment 910a to advance distally or proximally relative to the second sheath segment 910b. Similarly, the diameter of the distal opening surface 930 or distal opening 925 of the third sheath segment 910c can be sized to form a sliding fit with the outer proximal surface 920 of the second sheath segment 910b, such as to allow the second sheath segment 910b to advance distally or proximally relative to the third sheath segment 910c and/or the first sheath segment 910a. The telescoping vascular sheath 905 can include any number of sheath segments 910 that are slidably coupled together for forming a variety of length and diameter telescoping vascular sheaths, such as for performing a variety of procedures.

In some embodiments, the overall lengths of each sheath segment 910 can increase as the diameter of the outer proximal surface 920 decreases, as shown in FIGS. 4A and 4B. For example, the first sheath segment 910a can have an overall length (e.g., from proximal end 916 to distal end 918) of approximately 80 centimeters (cm) to approximately 200 cm, the second sheath segment 910b can have an overall length of approximately 75 cm to approximately 120 cm, the third sheath segment 910c can have an overall length of approximately 65 cm to approximately 85 cm, the fourth sheath segment 910d can have an overall length of approximately 25 cm to approximately 45 cm, and the fifth sheath segment 910e can have an overall length of approximately 5 cm to approximately 30 cm. The overall lengths of the sheath segments 910 can vary without departing from the scope of this disclosure. For example, the telescoping vascular sheath 905 and/or sheath segments 910 can include shorter lengths than the examples provided herein, such as for use with carotid access.

In some embodiments, the diameter of the distal opening 925 is approximately 0.010 inch (in) to approximately 10 French (F). For example, the diameter of the distal opening 925 of the first sheath segment 910a can be approximately 0.010 in to approximately 0.025 in, the diameter of the distal opening 925 of the second sheath segment 910b can be approximately 3 F to approximately 5 F, the diameter of the distal opening 925 of the third sheath segment 910c can be approximately 0.05 in to approximately 0.08 in, the diameter of the distal opening 925 of the fourth sheath segment 910d can be approximately 0.08 in to approximately 0.1 in, and the diameter of the distal opening 925 of the fifth sheath segment 910e can be approximately 7 F to approximately 10 F. Other diameters of the distal opening 925 are within the scope of this disclosure, such as approximately 20 F.

As shown in FIG. 4B, the sheath segment 910 that is positioned closest to the longitudinal axis L of the telescoping vascular sheath 905 (e.g., the first sheath segment 910a) can include a smallest diameter sheath segment passageway 915 having a segment passageway diameter that is smaller compared to segment passageway diameters of the other sheath segments 910 of the telescoping vascular sheath 905. Furthermore, since the first sheath segment 910a is positioned closest to the longitudinal axis L (e.g., no other sheath segment 910 is positioned within the first sheath segment 910a), the sheath segment passageway 915 of the first sheath segment 910a can define the working passageway 906 of the telescoping vascular sheath 905.

For example, the working passageway 906 can provide a safe and effective passageway for inserting and/or removing one or more of a device and a material to and from, respectively, the vasculature. The telescoping vascular sheath 905 is modular and adjustable such that the first sheath segment 910a (e.g., the sheath segment 910 that is closest to the longitudinal axis L of the telescoping vascular sheath 905), can be any one of the plurality of concentric sheath segments 910 thereby allowing the telescoping vascular sheath 905 to be customizable to have a working passageway 906 that is appropriately sized for a procedure.

As shown in FIGS. 4B and 4C, the proximal end 916 of at least one of the sheath segments 910 can include a port 950 with a fluid passageway 952 extending therethrough and in fluid communication with the segment passageway 915. The fluid passageway 952 can allow for fluid passage into and/or out of one or more of the sheath segment passageway 915 and/or the working passageway 906. The port 950 can extend out from a side of the sheath segment 910 and telescoping vascular sheath 905. The ports 950 and/or other proximal features of each sheath segment 910 can also provide stops or limitations for travel of at least one adjacent sheath segment 910, such as to prevent the proximal end 916 of a smaller diameter sheath segment 910 from dropping into the sheath segment passageway 915 of a larger diameter sheath segment 910. The port 950 can allow for administration of fluids (e.g., saline, contrast, drugs, etc.) into the inner lumen and/or the removal of fluids and/or tissue (e.g., blood, thrombus) from the inner lumen of a vessel.

In some embodiments, the telescoping vascular sheath can include a valve 960 positioned at a proximal end 916 of one or more sheath segments 910. For example, the valve 960 can include one or more of a hemostasis valve, rotating hemostasis valve (RHV), Tuohy Borst valve, luer lock, and/or other similar valves. For example, the valve 960 can be permanent, removable, and/or replaceable. In some embodiments, the valve 960 of one sheath segment 910 (e.g., the first sheath segment 910a) can lock with the valve 960 of an adjacent sheath segment 910 (e.g., the second sheath segment 910b). Such locking can enable at least two sheath segments 910 of the telescoping vascular sheath 905 to lock together and move as a single unit. In some embodiments, the valves 960 can enable the introduction, advancement, retraction, or removal of smaller sheath segments 910. For example, the valves 960 may be able to lock onto an adjacent sheath segment 910 to fix relative motion (axial, longitudinal). In some embodiments, one or more valves 960 can include an eyelet that allow suturing to patient skin or other items.

In some embodiments, one or more sheath segments 910 can be made out of a material that can allow the sheath segments 910 to be viewed under radiography and/or ultrasound. In some embodiments, one or more sheath segments 910 can include radiopaque markers (e.g., radiopaque bands) that can be viewed under radiography. In some embodiments, the sheath segments 910 can each have slight radiopacity, which can allow the telescoping vascular sheath 905 to become progressively more visible when more sheath segments 910 are concentrically coupled. In some embodiments, one or more sheath segments 910 can include a scored echogenic air pocket to enhance visibility under ultrasound. In some embodiments, one or more sheath segments 910 can include depth marking to allow a user to monitor advancement of the telescoping vascular sheath into tissue and/or a vessel. In some embodiments, one or more sheath segments 910 can include a balloon or other support feature that provides vascular occlusion and/or enables flow reversal.

FIG. 4E illustrates an embodiment of a sheath segment 1010 including an inflatable balloon 1011 positioned adjacent a distal end 918. For example, the balloon 1011 can be inflated once the distal end 918 is in a desired position within a vessel, such as for providing vessel occlusion. For example, the balloon 1011 can expand circumferentially or non-circumferentially around and/or relative to the proximal section of the respective sheath segment 1010 at one or more positions along the length of the sheath segment 1010. As such, although the balloon 1011 is shown in FIG. 4E as being positioned adjacent the tapered distal section 914, the balloon 1011 can be positioned along any part of the length of the sheath segment 1010 for assisting with one or more of a variety of procedures. Furthermore, the sheath segment 1010 including the balloon 1011 can have any number of a variety of shapes and sizes, including any of the lengths and diameters disclosed herein. The balloon 1011 can be made out of a variety of materials, including one or more of a biocompatible material and elastomeric material (e.g., polyurethane, silicone, etc.).

The outer dimensions of the telescoping vascular sheath 905 can be modified and customized in order to size the telescoping vascular sheath 905 such that it is appropriately sized for one or more procedures, such as for performing atraumatic insertion and positioning of the telescoping vascular sheath into one or more of a variety of vasculature. As such, the telescoping vascular sheath can be modified and customized to have outer dimensions and inner dimensions that are safe and effective for performing one or more procedures in one or more of a variety of vasculature.

As shown in FIG. 4D, during use, the first sheath segment 910a can be positioned most distally and have a smallest diameter proximal section 912 and smallest diameter distal opening 925. For example, the distal opening 925 of the first sheath segment 910a can be sized to have a sliding fit with a guidewire 611 for assisting with appropriately steering the distal end 918 of the telescoping vascular sheath 905 into and along a vessel. As also shown in FIG. 4D, the second sheath segment 910b can have a distal opening 925 that is approximately the same diameter as the proximal section of the first sheath segment 910a. For example, the second sheath segment 910b can have dimensions that allow the second sheath segment 910a to perform effective dilation.

Furthermore, the tapered distal sections 914 of each sheath segment 910, including the tapered distal wall 924 of each sheath segment 910, can facilitate atraumatic entry of each sheath segment 910 into the vessel V. For example, the tapered distal wall 924 can include a variety of lengths for achieving one or more of a smooth and gradual increase in diameter along the length of the telescoping vascular sheath 905. In some embodiments, the wall thickness of one or more sheath segments 910 can change along a length, such as gradually reduce in wall thickness towards the distal end 918, while maintaining a consistent diameter of the sheath segment passageway, to thereby form the tapered distal section 914.

The distance between the distal end 918 of the first sheath segment 910a and the distal end 918 of the second sheath segment 910b can be adjusted by sliding the first and second sheath segments relative to each other. Additionally, the first sheath segment 910a can be proximally retracted and removed from the telescoping vascular sheath 905, thereby leaving the second sheath segment 910b to be the most distal sheath segment 910. Similar features and functions can be achieved with any of the sheath segments 910 of the telescoping vascular sheath 905. For example, the second sheath segment 910b can be translated proximally and removed to thereby leave the third sheath segment 910c as the most distal sheath segment 910. For example, the third sheath segment 910c can assist with performing aspiration and, once the third sheath segment 910c is removed, the fourth sheath segment 910d can assist with performing guide catheter functions. As such, a single telescoping vascular sheath 905 can include a plurality of sheath segments 910 that can assist with performing a variety of aspects of a procedure without requiring removal and replacement of the telescoping vascular sheath 905 (only parts of sheath segments 910 of the telescoping vascular sheath 905, as appropriate). This can reduce procedure time and costs, as well as improve safety and recovery of the patient.

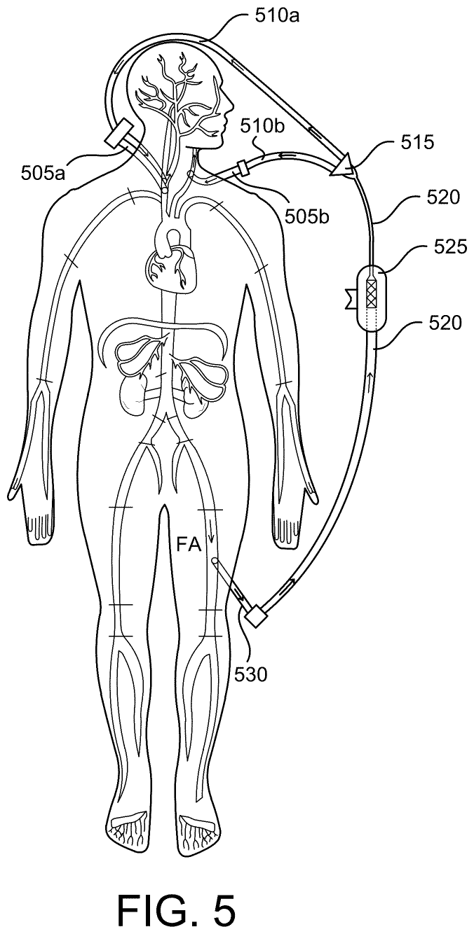

FIG. 5 is a schematic illustration of a blood flow system including a first arterial access device 505a that accesses the right common carotid artery (RCCA) and a second arterial access device 505b that accesses the left common carotid artery (LCCA) both via a transcervical approach. The system further includes an arterial return device 530 that communicates with an artery such as a femoral artery (FA). Each of the arterial access devices 505 and the arterial return device 530 has an internal lumen that can be positioned (surgically via a cut-down or percutaneously) to communicate with the respective artery.

For example, the arterial access devices 505 can be an access system 110, such as shown in FIG. 1, and can include all or a subset of the components of the access system 110, including one or more of a distal sheath 605, a proximal extension 610, a flow line 615, an adaptor, a Y-connector 620, and a hemostasis valve 625. The blood flow system further includes a first shunt 510a fluidly connected to the arterial access device 505a and configured to shunt blood flow from the femoral artery to the arterial access device 505a and into the RCCA. The blood flow system further includes a second shunt 510b fluidly connected to the arterial access device 505b and configured to shunt blood flow from the femoral artery to the second arterial access device 505b and into the LCCA. In some embodiments, one or more of the first arterial access device 505a, the second arterial access device 505b, and the arterial return device 530 can include an embodiment of the telescoping vascular sheath 905 described herein.

With reference still to FIG. 5, the arterial return device 530 is in fluid communication with the femoral artery FA (or another artery) and is configured to receive blood flow from the FA. The arterial return device 530 is fluidly connected to a return shunt 520 through which blood flows from the respective artery (e.g., the femoral artery in FIG. 5) via the arterial return device 530 toward the arterial access devices 505. A splitter 515 splits the single return shunt 520 into separate flow lumens of the first shunt 510a and the second shunt 510b. A flow control element 525 is coupled to the return shunt 520 and is configured to control blood flow (such as a rate of flow) through the return shunt 520. A blood filter can also be coupled to the shunt 520.

In use, blood flows out of the femoral artery into the return shunt 520 via the arterial return device 530. The flow control element 525 enables control of the flow of blood through the return shunt 520 as it flows toward the splitter 515. Blood then flows through the first shunt 510a and the second shunt 510b toward the arterial access device 505a and arterial access device 505b, respectively, where blood can then flow into the respective artery. The LCCA and the RCCA can be occluded such as via an expandable element (such as an expandable balloon) on the respective arterial access device 505a and arterial access device 505b or by using a clamp.

While these descriptions contain many specifics, these should not be construed as limitations on the scope of an invention that is claimed or of what may be claimed, but rather as depictions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore, the spirit and scope of the appended claims should not be limited to the description of the embodiments contained herein.

1-38. (canceled)

39. A telescoping vascular sheath for insertion into a vessel, comprising:
a plurality of sheath segments each having a tapered distal section and a proximal section, each tapered distal section including a distal opening surface and each proximal section including an outer proximal surface, each sheath segment of the plurality of sheath segments being slidably engaged with at least one adjacent sheath segment along one or more of the distal opening surfaces and the outer proximal surface; and
a working passageway extending through the plurality of sheath segments and along a longitudinal axis of the telescoping vascular sheath, the working passageway providing access to an inner lumen of the vessel.

40. The telescoping vascular sheath of claim 39, wherein each sheath segment of the plurality of sheath segments is independently moveable along the longitudinal axis.

41. The telescoping vascular sheath of claim 39, wherein the proximal section includes a tubular shape that extends a distance along the longitudinal axis.

42. The telescoping vascular sheath of claim 39, wherein the tapered distal section includes a distal opening defined by the distal opening surface.

43. The telescoping vascular sheath of claim 39, wherein the distal opening surface is approximately linear and parallel to the longitudinal axis.

44. The telescoping vascular sheath of claim 39, wherein the working passageway is defined by a first sheath segment of the plurality of sheath segments, the distal opening surface of the first sheath segment being smaller in diameter compared to distal opening surfaces associated with each of the other sheath segments of the plurality of sheath segments.

45. The telescoping vascular sheath of claim 44, wherein the distal opening surface of a second sheath segment of the plurality of sheath segments forms a sliding fit along the proximal section of the first sheath segment.

46. The telescoping vascular sheath of claim 39, wherein the tapered distal section provides atraumatic insertion of the telescoping vascular sheath into tissue and the inner lumen of the vessel.

47. The telescoping vascular sheath of claim 39, wherein a first sheath positioned closest to a longitudinal axis of the telescoping vascular sheath includes the proximal section having a smaller diameter and an overall length that is longer compared to other sheath segments of the telescoping vascular sheath.

48. The telescoping vascular sheath of claim 39, wherein an outer sheath segment of the plurality of sheath segments is positioned furthest away from the longitudinal axis of the telescoping vascular sheath, the outer sheath segment including a first proximal section that is greater in diameter and shorter in length compared to proximal sections associated with each of the other sheath segments of the plurality of sheath segments.

49. The telescoping vascular sheath of claim 39, further comprising a valve positioned at a proximal end of at least one sheath segment of the plurality of sheath segments.

50. The telescoping vascular sheath of claim 39, further comprising a port positioned at a proximal end of at least one sheath segment of the plurality of sheath segments, the port including a fluid pathway that is in fluid communication with the working passageway.

51. The telescoping vascular sheath of claim 39, wherein at least one sheath segment of the plurality of sheath segments includes an inflatable balloon.

52. The telescoping vascular sheath of claim 51, wherein the inflatable balloon expands radially out from the proximal section when in an inflated state.

53. The telescoping vascular sheath of claim 39, wherein at least one sheath segment of the plurality of sheath segments provides atraumatic dilation of tissue and atraumatic dilation into the vessel.

54. A method of a telescoping vascular sheath, the method comprising:
advancing a first sheath segment of the telescoping vascular sheath into an inner lumen of a vessel;
advancing a second sheath segment of the telescoping vascular sheath into the inner lumen of the vessel, wherein the telescoping vascular sheath comprises:
a plurality of sheath segments each having a tapered distal section and a proximal section, the plurality of sheath segments including the first sheath segment and the second sheath segment, each tapered distal section including a distal opening surface and each proximal section including an outer proximal surface, each sheath segment of the plurality of sheath segments being slidably engaged with at least one adjacent sheath segment along one or more of the distal opening surfaces and the outer proximal surface, and
a working passageway extending through the plurality of sheath segments and along a longitudinal axis of the telescoping vascular sheath, the working passageway providing access to the inner lumen of the vessel; and
slidably translating the first sheath segment relative to the second sheath segment.

55. A blood flow system, comprising:
a first arterial access device for accessing a first artery;
a second arterial access device for accessing a second artery;
an arterial return device for accessing a third artery, and
wherein the arterial return device is in fluid communication with the first arterial access device and the second arterial access device.

56. The blood flow system of claim 55, wherein one or more of the first arterial access device, the second arterial access device, and the arterial return device comprise a telescoping vascular sheath.

57. The blood flow system of claim 55, further comprising a splitter that directs fluid flow from the arterial return device to both the first arterial access device and the second arterial access device.

58. The blood flow system of claim 57, further comprising one or more of a flow control element that controls rate of flow between the arterial return device and the splitter.
